(19) United States
(12) Patent Application Publication
(10) Pub. No.: US 2011/0129812 A1
(43) Pub. Date: Jun. 2, 2011

(54) METHOD AND APPARATUS FOR INJECTION OF CO2 OR STACK GASSES TO INCREASE ALGAL BIOMASS PRODUCTION

(76) Inventors: Walter H. Adey, Reedville, VA (US); Erik T. Adey, Clear, AK (US)

(21) Appl. No.: 12/950,422

(22) Filed: Nov. 19, 2010

Related U.S. Application Data
(60) Provisional application No. 61/263,168, filed on Nov. 20, 2009.

(51) Int. Cl. C12Q 3/00; C12M 1/00; C12M 1/12; C12N 1/12

(52) U.S. Cl. 435/3; 435/289.1; 435/297.1; 435/305.1; 435/257.1

(57) ABSTRACT

An algal production system that uses CO2 injections to promote the growth of algae. The system includes an algal growth medium within a floway for channeling water. A fluid diffuser also resides in the floway in proximity to the algal growth medium. The fluid diffuser injects CO2 into the water in the floway. The system further includes a detector for monitoring the pH levels of the water and a controller, which based on the measured pH levels, determines when and how much CO2 to inject into the water.

### CROSS-REFERENCE TO RELATED APPLICATIONS

This application claims the benefit of U.S. provisional application 61/263,168, filed on Nov. 20, 2009, the subject matter of which is incorporated herein by reference in its entirety.

### BACKGROUND

The following described method and apparatus relates to the algal production technology which was conceived and developed over a period of about 30 years and patented as U.S. Pat. Nos. 4,333,263, issued Jun. 8, 1982; 4,966,096, issued Oct. 30, 1990; 5,097,795, issued Mar. 24, 1992; 5,851,398, issued Dec. 22, 1998; and 5,715,774, issued Feb. 10, 1998; the disclosures of which are incorporated herein by reference. Existing large-scale algal production systems marketed under the brand Algal Turf Scrubber®, or ATS systems include in-ground troughs or “floways.” Many small-scale algal production systems have been utilized for research and aquaria under the existing patents referenced above.

As in most photosynthetic systems, algae require carbon to complete the chemical process of photosynthesis with production of organic material. The carbon is taken from carbon dioxide (CO2) and bicarbonate (HCO3) in the ambient water and is effectively a nutrient, the concentration of which will affect the rate of productivity or biomass building.

In the modern era of concern about climate change and excess CO2 release into the atmosphere, there exists a need for disposal or reutilization of CO2 produced as a result of industrial operations, in particular electrical generation at coal and oil fired power plants.

### SUMMARY

This method and apparatus described herein provides an algal production system. The system may include a method and apparatus for introduction of CO2 to affect the production of algal biomass. The system may utilize carbon dioxide (CO2) from industrial operations to reduce release into the atmosphere.

In one aspect, the system includes a floway for channeling water from a water source, an algal growth medium arranged within the floway, and a fluid diffuser arranged in proximity to the algal growth medium. The fluid diffuser is configured to diffuse fluid from a fluid source into the water to promote the growth of algae on the algal growth medium. The fluid source includes a gas source or a fluid source, such as, a gas dissolved in a liquid, such as, water. The fluid may include a nutrient which may be CO2 gas.

In another aspect, the system includes a controller for controlling the fluid supplied by the fluid source and the water supplied from the water source. A detector is coupled to the controller, the detector measuring the pH level of the water and sending the measurements to the controller. The controller uses the pH level measurements to determine the amount of water supplied by the water source and the amount of fluid supplied by the gas source. pH should generally be maintained between about 7.5 to about 8.5 for some algae and growing conditions.

In another aspect, the fluid diffuser of the system includes a plurality of tubes coupled to a mat. The floway has a plurality of ridges arranged in parallel along a surface with a space between each ridge, and at least one tube of the plurality of tubes is located in each space.

In another aspect, the fluid diffuser of the system is located within a depression in the floway. The depression runs parallel to a length of the floway, or may run at an angle, such as perpendicular to a length of the floway.

In another aspect, the floway of the system includes a plurality of segments connected together. The depression may be located at a connection of one segment to another.

In another aspect, a method of producing algae includes the steps of providing a floway having an algal growth medium arranged within the floway, channeling water along the floway, and diffusing fluid into the water of the floway by use of a fluid diffuser arranged on the upper surface of the algal growth medium, configured to diffuse gas, such as CO2, from a fluid source into the water to promote the growth of algae on the algal growth medium.

In another aspect, a method of producing algae includes the steps of providing a floway having an algal growth medium arranged within the floway, channeling water along the floway, and diffusing gas into the water of the floway by the addition of carbonated fluid at one or more points along the floway to promote the growth of algae on the algal growth medium. Carbonated water may be produced external to the system by injection of CO2 or stack gasses from a fluid source into water in a container or tubing, and then introduced via tubing to the floway.

In another aspect, the method includes the step of controlling the flow of water into the floway and the flow of fluid into the water by use of a controller. The pH level of the water is measured and provided to the controller. The controller uses the pH level measurements to determine the amount of water supplied to the floway or the amount of fluid supplied to the water.

### BRIEF DESCRIPTION OF THE DRAWINGS

FIG. 1 is a schematic view of a system including a tube mat for introducing gas into water near algal growth of an algal production system according to a preferred embodiment.

FIG. 2 is a bottom end view of the tube mat of FIG. 1.

FIG. 3 is a manifold end view of the tube mat of FIG. 1.

FIG. 4 is a cross section elevation view of the tube mat of FIG. 1 arranged within a floway.

FIG. 5 is a side view of gas tubes arranged within depressions of a floway of an algal production system according to another preferred embodiment.

FIG. 6 is a plan view of a gas tube arranged within a junction between two floways of an algal production system according to another preferred embodiment.

FIG. 7. is a view taken along section line VII-VII of FIG. 6.

### DETAILED DESCRIPTION OF PREFERRED EMBODIMENTS

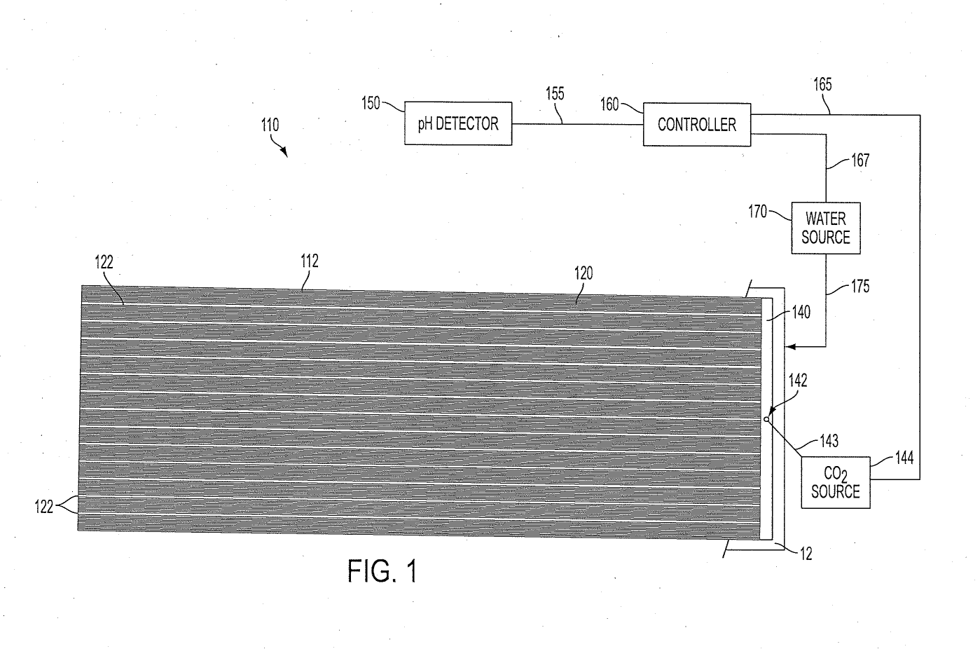

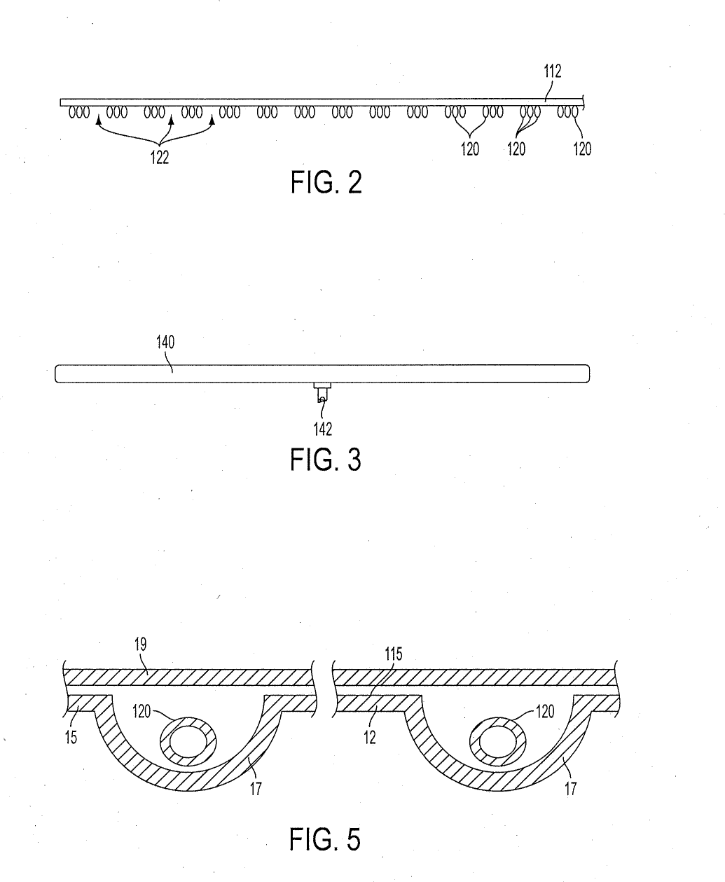

Referring now to the drawings, where like reference numerals designate like elements, there is shown in FIG. 1 an algal production system 110 according to a preferred embodiment. The system 110 provides a method and apparatus for introduction of gasses, of which CO2 is of primary interest in the illustrated embodiment, to the water adjacent to the cell walls of algal filaments via membrane systems located in proximity to algal growth screens on algal production systems such as Algal Turf Scrubber® systems. Introduction of other substances may also be desirable, such as gaseous or liquid phosphorus or nitrogen ion may also be accomplished with membranes, for example. These membranes are readily available on the market today for scientific and industrial uses, and exist in a variety of conformations related to use.

Injection of CO2 and/or stack gasses into waters that will be treated by algal remediation satisfies the need for disposal of the CO2, while presenting the opportunity to increase production of algal biomass.

Simple injection into passing water flow via a bubbler system and air stones could be utilized, provided that losses to the atmosphere are limited. Bubble walls do not present gasses to the algal cells in an effective manner, reducing uptake by the cells as the water passes. Surface area relative to volume is lower as bubble size increases, suggesting that smaller bubbles would be more effective in diffusing CO2 into solution. Bubblers producing extremely fine bubbles may be useful in introducing CO2 and other gasses to an algal growth medium, provided the bubblers are of sufficient length and of sufficiently small size to be maintained in close proximity to algal cells without undue loss of CO2 to the atmosphere.

Membrane technology allows introduction of selected material on the atomic or molecular level, obviating the process of diffusion across the air/water boundary in the case of gas bubbles. Direct diffusion using submerged membranes will minimize losses of gasses to the atmosphere, and maximize availability to algal cells. Membranes exist on the market in a wide variety of physical conformations, and have designed chemical compositions for uses such as reverse osmosis and kidney dialysis, and can permit or prevent the passage of gasses or liquids in a range of sizes from atomic to large complex molecules. Membranes suited to the selective diffusion of CO2 could be used for application to algal cells, in tubular conformation, in a range of sizes suitable for the algal growth medium. Sheet membranes could be installed beneath algal growth media, fed by pressure connection through the bottom of the algal growth units, but would likely be harder to maintain. Testing has shown that gasses such as CO2 and stack gasses are easily introduced to water using membrane technology. While large pore sizes do diffuse the gasses into the water, there may be some loss to the atmosphere due to bubble formation. The preferred pore size would be less than one micron. For ideal algal uptake of the CO2 gas diffused into water, the pore size should be less than one tenth of a micron. Pore size would be larger for a liquid such as carbonated water or a nutrient solution such as nitrogen or phosphorus solutions.

Since CO2 and HCO3 are part of the carbonate system in an aqueous environment, pH measurement may be used for controlling CO2 and/or water flow rates to control the rate of addition of carbon to the algal photosynthetic/productivity process.

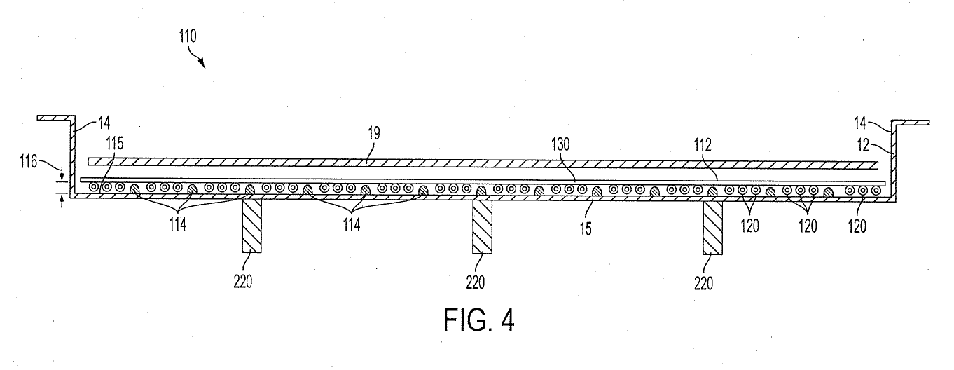

A preferred configuration illustrated in the FIGS. includes a number of small hollow membrane tubes 120 made of CO2 specific membrane material and laid in an enclosing mesh 130 such that they form a sheet mat 112 approximately forty-two inches wide and ten feet long. A gas supply manifold 140 joins all the membrane tubes 120 at one end, with a pressure hose connection 142 entering the manifold 140 at an appropriate location to suit application. CO2 gas is fed into the manifold 140 and distributed through the inside of the hollow membrane tubes 120. The gas molecules inside the tubes migrate, driven by the pressure differential, through the wall of the tubes to outside the tubes to enter the water in proximity to the algal filaments on the algal growth screen 19. This sheet 112 of membrane tubes 120 would be laid underneath or in proximity to the algal growth medium 19 within the floway 12 (FIG. 4), and connection would be supplied via a conduit 143 from a source 144 of CO2. The source 144 provides the CO2 in gas form. The algal growth medium could be a plastic mesh available from netting suppliers. The plastic mesh should have a size range, depending upon the algae involved, on the order of from about one millimeter to about one centimeter.

In an alternative embodiment, the source 144 may provide carbonated water that contains CO2. In this embodiment, the fluid flows into the membrane tubes 120 and is distributed by the membrane tubes 120 into the water in proximity to the algal filaments on the algal growth medium 19. The carbonated water mixes with the water to provide the CO2 to the algal growth.

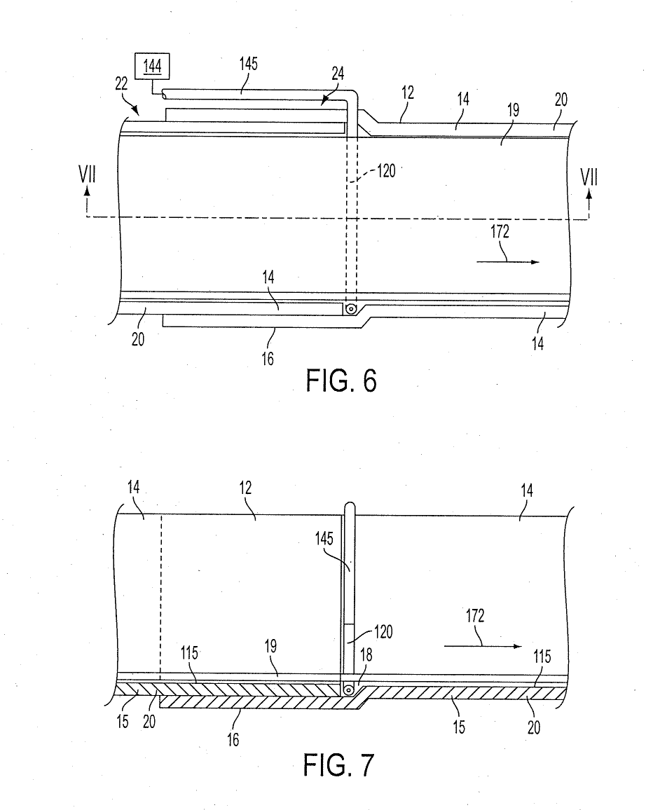

This system can be applied in any algal production systems including ATS systems. An algal production system may include a trough or floway 12 having a floway bottom 15 and floway sides 14. The floway 12 may be supported at an incline for water to flow down the incline. The water may be provided in surges from a bucket (not shown) that functions like a tipping bucket as described in U.S. Pat. No. 4,966,096. The floway 12 may be constructed from modular pieces or floway “trays” or “segments” 20 arranged end to end and supported by suitable supports 220. As shown in the illustrated embodiment of FIG. 6, each of the floway segments 20 has an upper segment end 24 and a lower segment end 22. Within the floway 12, there are several ridges 114 on the floway bottom 15 integral to the fiberglass of the floway 12 and extending in a longitudinal direction. The ridges 114 preferably have a height 116 of approximately one quarter to one half inch above the top surface 115 of the tray bottom 15. The sheet mat 112 for CO2 injection fits over these ridges 114 such that the membrane tubes 120 rest in the low areas between the ridges 114, and the enclosing mesh 130 holds the membrane tubes 120 in position relative to each other between the ridges 114, and positioned below the algal growth medium 19. The membrane tubes 120 may or may not be attached to the mesh 130. In another embodiment, the membrane tubes 120 may be woven into the mesh 130 to form the sheet 112. In another embodiment, the membrane tubes 120 may be incorporated into the algal growth matrix itself, such as by gluing, welding, or weaving.

The gas pressure fitting 142 on the manifold 140 is located at a ninety degree angle to the manifold 140 and penetrates through the bottom 15 of the floway 12 such that connection to the gas source can be accomplished beneath the floway 12. Each of the membrane sheets 112 is about the same length as each floway tray segment 20 of the fiberglass floway 12 in the illustrated embodiment. Thus, there will be a maximum number of sheets 112 equal to the number of floway segments 20. Not all floway segments 20 will necessarily need to have installed membrane sheets, and gas flow to each sheet can be adjusted to accomplish maximum algal growth enhancement, allowing adaptation to particular desired water and algal growth requirements. pH measurement will be used to control either water flow rate or CO2 input pressure, or both, to maximize algal productivity and nutrient removal relative to the typical need to allow pH elevation near the end of an ATS floway where zonal precipitation of phosphorus, heavy metals, and other minerals would occur (see U.S. Pat. No. 5,851,398).

The pH detector 150 senses the pH level of the water from one or more locations in or downstream along the floway 12. The pH information is communicated 155 to the controller 160, which processes the pH information, communicates 165 to control the CO2 gas pressure via the source 144, and communicates 167 to control the water flow via the water source 170. The communications may be wired or wireless or other media and computers may be used to effect communications.

FIG. 5 shows another embodiment of the system 110 with the membrane tubes 120 laid in one or more depressions 17 in the floway 12. The depressions 17 are wells that are integrally manufactured along the bottom of the floway 12. The depressions 17 may run parallel to the sides 14 of the floway 12 along the length of the floway 12. Alternatively, the depressions 17 may run perpendicular to the sides 14 of the floway 12 along the width of the floway 12. The depressions 17 have sufficient depth so that the membrane tubes 120, when placed within the depressions 17 are substantially or wholly located below the top surface 115 of the floway bottom 15 and submerged beneath the algal growth medium 19 within the floway 12. In another embodiment, the membrane tubes 120 within the depressions 17 may not be wholly below the top surface 115 of the floway bottom 15 but are still be located below the algal growth medium 19. Locating the membrane tubes 120 within the depressions 17 keeps the membrane tubes 120 substantially below the plane of the top surface 115 of the floway bottom 15 so as to lessen any contact or interference with the growth medium 19 as the growth medium 19 is maintained, removed or serviced by an harvesting apparatus to harvest algal growth.

FIGS. 6 and 7 show another embodiment of the system 110 with the membrane tubes 120 laid in a groove 18 between adjoining segments 20 that form the floway 12. Each segment 20 has a flange 16 at one end that couples the segment 20 to the non-flanged end of an adjacent segment 20. The groove 18 is formed in the bottom 15 of the floway 12 where the two segments 20 are coupled by the flange 16. The membrane tube 120 is placed within the groove 18 to be located substantially wholly beneath the top surface 115 of the floway bottom 15 and beneath the algal growth membrane 19. The membrane tube 120 within the groove 18 is perpendicular to the sides 14 of the floway 12 and to the direction of the water flow 172. The membrane tube 120 is coupled to a supply line 145, which is coupled to the conduit 143 and the CO2 source 144 (FIG. 1). The supply line 145 of the illustrated embodiment runs inside the vertical groove in the floway sidewall 14 formed by the flange 16, and then along the top or the side of the floway sidewalls 14 to the source 144 of gas. The supply line 145 may also serve as a supply line to additional membrane tubes 120 that may be positioned in the floway 12. For example, multiple membrane tubes 120 running lengthwise within the floway 12 may connect to the supply line 145.

The membrane tubes 120 are placed in any number of flange grooves 18 or depressions 17 along the floway 12 to inject CO2 into the water in the floway 12 to suit the application and water chemistry. A single membrane tube 120 may be used in a single location or multiple locations along the floway 12. Alternatively, multiple membrane tubes 120 may be used in a single location or multiple locations along the floway 12. Additionally, the membrane tubes 120 may be located in one or more selected areas the floway 12. The floway 12 may have the membrane tubes 120 located in the grooves 18 as shown in FIGS. 6 and 7, as well as having the membrane tubes 120 within the depressions 17 as shown in FIG. 5.

The injection of CO2 affects the pH level and/or increases the available carbon in the water. The injection of CO2 into the water from the tubes 120 shown in FIGS. 5-7 is controlled by the controller 160 based on information provided by the detector 150 as previously described. The controller 160 is able to maintain desired pH levels along the floway 12 within a range suitable for the prescribed water, algae species and production requirements. The pH level can be adjusted to control the type of algae that will grow within the floway 12. For example, in the brackish waters of the Chesapeake Bay, a desired pH level for the growth of the algae including those in the genera Berkeleya and Melosira is preferably in the range of from about 7.0 to about 8.5. The desired range of pH level will depend on the specific species of algae and the composition of the water, such as the salinity of seawater, brackish water, and fresh water.

Additionally, the pH level and/or the available carbon in the water may be controlled to obtain certain characteristics of the algal growth. For example, more than one species of algae may grow on the growth medium 19. The pH level may be controlled to vary the relative proportions of the algae species. Additionally, the pH and/or the available carbon in the water may be controlled to vary characteristics of specific algae growing on the growth medium 19.

It should be apparent that many modifications and variations of the preferred embodiments as hereinbefore set forth may be made without departing from the spirit and scope of the present invention. The specific embodiments described are given by way of example only. The invention is limited only by the terms of the appended claims.

What is claimed as new and desired to be protected by Letters Patent of the United States is:

1. An algal production system comprising:
a floway for channeling water;
an algal growth medium within the floway; and
a fluid diffuser arranged within the floway and adjacent to the algal growth medium, the fluid diffuser configured to diffuse fluid into the water.

2. The system of claim 1, further comprising a fluid source coupled to the fluid diffuser, the fluid source supplying fluid to the fluid diffuser.

3. The system of claim 2, wherein the fluid supplied by the fluid source is a carbonated fluid.

4. The system of claim 3, wherein the fluid supplied by the fluid source is carbon dioxide gas.

5. The system of claim 4, wherein the fluid diffuser is a membrane with pores.

6. The system of claim 5, wherein the pores are less than or equal to 0.1 microns.

7. The system of claim 2, further comprising a controller for controlling the fluid supplied by the fluid source.

8. The system of claim 7, further comprising a water source for supplying the water to the floway, the water source being controlled by the controller.

9. The system of claim 7, further comprising a detector coupled to the controller, the detector measuring the pH level of the water and sending the measurements to the controller.

10. The system of claim 9, wherein the controller uses the pH level measurements to determine the amount of water supplied by the water source.

11. The system of claim 9, wherein the controller uses the pH level measurements to determine the amount of fluid supplied by the fluid source.

12. The system of claim 1, wherein the fluid diffuser includes a tube.

13. The system of claim 12, wherein the fluid diffuser includes a plurality of tubes.

14. The system of claim 13, wherein the plurality of tubes are coupled to a matt.

15. The system of claim 13, wherein the floway has a plurality of ridges arranged in parallel along a surface with a space between each ridge, wherein at least one tube of the plurality of tubes is located in each space.

16. The system of claim 1, wherein the fluid diffuser is located within a depression in the floway.

17. The system of claim 16, wherein the depression runs parallel to a length of the floway.

18. The system of claim 16, wherein the depression runs perpendicular to a length of the floway.

19. The system of claim 16, wherein the floway comprises a plurality of tray segments connected together, wherein the depression is located at a connection of one segment to another segment.

20. The system of claim 1, wherein the fluid diffuser includes a mat.

21. The system of claim 1, wherein the fluid diffuser includes a membrane.

22. The system of claim 1, wherein the fluid diffuser includes an air stone.

23. A method of producing algae comprising the steps of:
providing a floway having an algal growth medium arranged within the floway;
channeling water along the floway; and
diffusing fluid into the water of the floway by use of a fluid diffuser arranged in proximity to the algal growth medium.

24. The method of claim 23, further comprising the step of controlling the flow of fluid into the water by use of a controller.

25. The method of claim 23, further comprising controlling the flow of water into the floway by use of a controller.

26. The method of claim 23, further comprising the steps of measuring the pH level of the water using a detector and providing the measurements to a controller.

27. The method of claim 24, further comprising the step of using the pH level measurements to determine the amount of water supplied to the floway or the amount of fluid supplied to the water.

28. The method of claim 23, wherein the fluid diffuser comprises a plurality of tubes.

29. The method of claim 23, wherein the step of providing the floway comprises connecting a plurality of tray segments together to create the floway.

30. The method of claim 23, wherein the fluid diffuser includes a mat.

31. The method of claim 23, wherein the fluid diffuser includes a membrane.

32. The method of claim 31, wherein the membrane has pores less than or equal to 0.1 microns.

33. The method of claim 23, wherein the fluid diffuser includes an air stone.

34. The method of claim 23, wherein the fluid diffused into the water is a carbonated fluid.

35. The method of claim 23, wherein the fluid diffused into the water is gaseous carbon dioxide.

36. The method of claim 23, wherein the fluid diffused into the water increases the available carbon in the water.

37. The method of claim 23, wherein the fluid diffused into the water affects the pH level of the water.
